(19) United States
(12) Patent Application Publication
(10) Pub. No.: US 2025/0297998 A1
(43) Pub. Date: Sep. 25, 2025

(54) SYSTEM AND METHODS FOR MEASURING AIRBORNE GLYCOL

(71) Applicant: Grignard Pure LLC, Rahway, NJ (US)

(72) Inventors: Emile Etienne Grignard, III, Long Branch, NJ (US); Mark Lojek, Livingston Manor, NY (US)

(21) Appl. No.: 18/869,490

(22) PCT Filed: Jun. 2, 2023
(86) PCT No.: PCT/US2023/024299

§ 371 Date: Nov. 26, 2024

Related U.S. Application Data
(60) Provisional application No. 63/365,729, filed on Jun. 2, 2022.

(51) Int. Cl. G01N 33/00; G01N 15/06; G01N 15/10

(52) U.S. Cl. CPC G01N 33/0047; G01N 15/06; G01N 15/10; G01N 33/0032; G01N 33/0062

(57) ABSTRACT

An example system is configured to estimate a concentration of an airborne glycol in an environment. The system includes a processor, a display device, a first sensor configured to generate first sensor data representing a concentration of particles in the environment having a diameter less than a threshold diameter, a second sensor configured to generate second sensor data representing a temperature of the environment, and a third sensor configured to generate third sensor data representing a humidity of the environment. The processor is configured to receive the first sensor data, the second sensor data, and the third sensor data: estimate, based on the first sensor data, second sensor data, and the third sensor data, the concentration of the airborne glycol in the environment; and present, using the display device, a visual indication representing the concentration of the airborne glycol in the environment.

### CROSS-REFERENCE TO RELATED APPLICATIONS

This application claims the benefit of priority of U.S. Provisional Appl. No. 63/365,729 filed Jun. 2, 2022, the contents of which are incorporated by reference herein in its entirety.

### TECHNICAL FIELD

This disclosure relates to systems and methods for detecting airborne glycol, such as airborne triethylene glycol (TEG) and/or airborne propylene glycol (PG).

### BACKGROUND

An environment may contain airborne pathogens or surface-borne pathogens that can cause illness or harm to people within the environment. As an example, an environment may contain microbes (e.g., bacteria, fungi, etc.) and/or viruses that, if inhaled or otherwise come into contact with people within the environment, cause the people to suffer from a disease, allergies, or other adverse reaction.

### SUMMARY

A detection system is configured to determine the presence of and/or a concentration of airborne glycol in an environment. As an example, the detection system can be configured to determine the presence of and/or a concentration of airborne triethylene glycol (TEG), airborne propylene glycol (PG), or a combination thereof. In some implementations, the detection system can be configured to detect different forms of airborne glycol, such as vaporized glycol (e.g., vaporized TEG, vaporized PG, etc.) and aerosolized glycol (e.g., aerosolized TEG, aerosolized PG, etc.).

In some implementations, the detection system can be configured to detect the active ingredients of a sanitizing formulation that has been vaporized and/or aerosolized in the environment. As an example, a sanitization formulation can include an aqueous solution having TEG, PG, or a combination thereof. The sanitization formulation can be vaporized and/or aerosolized in the environment to neutralize airborne microbes, fungi, and/or viruses in the environment. Further, the detection system can be configured to monitor the concentration of airborne TEG and/or airborne PG in the environment, such that the airborne TEG and/or airborne PG can be maintained at a safe and efficacious concentration within the environment.

In some implementations, the detection system can be configured to determine the presence of and/or a concentration of airborne glycol in an environment based, at least in part, on sensor measurements representing (i) a concentration of airborne particulate matter in the environment having a diameter less than a particular threshold diameter (e.g., 2.5 μm, 10 μm, or some other threshold diameter), (ii) a temperature in the environment, and (iii) a humidity of the environment. For example, the detection system can obtain sensor measurements representing a concentration of airborne particulate matter in the environment having a diameter less than 2.5 μm, calibrate or adjust the sensor measurement based on the temperature and/or humidity of the environment, and output the calibrated and/or adjusted sensor measurement as an estimate of the concentration of airborne glycol in the environment. Further, in some implementations, the detection system can be configured to determine the presence of and/or a concentration of airborne glycol in an environment without use of a sensor that measures the presence and/or concentration of glycol through chemical processes, such as an electrochemical sensor, photoionization sensor, or a spectrometer.

One or more of the implementations described herein can provide various technical benefits. For example, implementations of the detection system described herein can allow users to determine the concentration of airborne glycol in an environment in a quick and efficient manner. This may be particularly useful, for example, in determining whether the concentration of airborne glycol in an environment is sufficiently high to neutralize airborne microbes, fungi, and/or viruses within the environment, while also sufficiently low to avoid adversely affecting people in the environment. Further, implementations of the detection system described herein can be used to detect airborne glycol without the use of chemical processes (e.g., without the use of electrochemical sensors or a photoionization sensors), which may otherwise increase the complex and/or cost of the detection system.

In an aspect, a system is configured to estimate a concentration of an airborne glycol in an environment. The system includes: a first sensor configured to generate first sensor data representing a concentration of particles in the environment having a diameter less than a threshold diameter; a second sensor configured to generate second sensor data representing a temperature of the environment; a third sensor configured to generate third sensor data representing a humidity of the environment; a display device; and one or more processors communicatively coupled to the first sensor, the second sensor, the third sensor, and the display device. The one or more processors are configured to: receive the first sensor data, the second sensor data, and the third sensor data; estimate, based on the first sensor data, second sensor data, and the third sensor data, the concentration of the airborne glycol in the environment; and present, using the display device, a visual indication representing the concentration of the airborne glycol in the environment.

Implementations of this aspect can include one or more of the following features.

In some implementations, the airborne glycol can include triethylene glycol (TEG) and propylene glycol (PG).

In some implementations, the airborne glycol can include vaporized TEG, aerosolized TEG, vaporized PG, and aerosolized PG.

In some implementations, the threshold diameter can be 10 μm.

In some implementations, the threshold diameter can be 2.5 μm.

In some implementations, presenting the visual indication representing the concentration of the airborne glycol in the environment can include at least one of: presenting a first visual indication responsive to determining that the concentration of the airborne glycol in the environment is less than a first threshold concentration, presenting a second visual indication responsive to determining that the concentration of the airborne glycol in the environment is greater than or equal to the first threshold concentration and less than a second threshold concentration, and presenting a third visual indication responsive to determining that the concentration of the airborne glycol in the environment is greater than or equal to a third threshold concentration.

In some implementations, the first threshold concentration can be 0.4 mg/m³ and the second threshold concentration can be 9.0 mg/m³.

In some implementations, estimating the concentration of the airborne glycol in the environment can include: multiplying the first sensor data by one or more calibration factors to obtain first calibrated sensor data, and estimating the concentration of the airborne glycol in the environment based on the first calibrated sensor data.

In some implementations, the one or more calibration factors can present a difference between an output of the first sensor and an output of a reference sensor configured to detect a concentration of particles in a reference environment having a diameter less than the threshold diameter.

In some implementations, the one or more calibration factors can include: a first calibration factor having a value of 0.23 or 0.4, and a second calibration factor having a value of 0.00075.

In some implementations, estimating the concentration of the airborne glycol in the environment can include: determining an adjustment value based on the second sensor data and the third sensor data, multiplying the adjustment value by an additional calibration factor to obtain a calibrated adjustment value, determining a sum of the first calibrated sensor data and the calibrated adjustment value, and estimating the concentration of the airborne glycol in the environment based on the sum of the first calibrated sensor data and the calibrated adjustment value.

In some implementations, estimating the concentration of the airborne glycol in the environment can include determining that the sum of the first calibrated sensor data and the calibrated adjustment value is the concentration of the airborne glycol in the environment.

In some implementations, the airborne glycol can include an aerosolized portion of the glycol, and a vaporized portion of the glycol. The first calibrated sensor data can represent a concentration of the aerosolized portion of the glycol in the environment, and the calibrated adjustment value can represent a concentration of the vaporized portion of the glycol in the environment.

In some implementations, the additional calibration factor can be 0.50 mg/m³.

In some implementations, the additional calibration factor can be in a range from 0.4 mg/m³ to 1.0 mg/m³.

In some implementations, the additional calibration factor can be in a range from 0.5 mg/m³ to 0.7 mg/m³.

In some implementations, the adjustment value can increase with an increase in the temperature of the environment.

In some implementations, the adjustment value can decrease with an increase in the humidity of the environment.

In some implementations, the one or more processors can be configured to: receive a user input comprising instructions to tare at least one of the first sensor data, the second sensor data, or the third sensor data, and responsive to receiving the user input, tare at least one of the first sensor data, the second sensor data, or the third sensor data.

In some implementations, the system can further include a transmitter configured to transmit, to a communications network, at least one of: the first sensor data, the second sensor data, the third sensor data, or data representing the concentration of the airborne glycol in the environment.

In some implementations, the transmitter can include a wireless transmitter.

In some implementations, the transmitter can be configured to transmit, to a glycol dispersal system via the communications network, at least one of the first sensor data, the second sensor data, the third sensor data, or the data representing the concentration of the airborne glycol in the environment. Further, the glycol dispersal system can be configured to regulate the concentration of the airborne glycol in the environment based on at least one of the first sensor data, the second sensor data, the third sensor data, or the data representing the concentration of the airborne glycol in the environment.

In some implementations, the system can also include comprising a housing. The housing can at least partially enclose the first sensor, the second sensor, and third sensor, and the one or more processors.

In some implementations, the system can further include a power supply at least partially enclosed by the housing.

In some implementations, the system can further include a connector configured to electrically couple the system to an external power supply.

In some implementations, the display device can include at least one of: one or more indicator lights, or a display panel.

In another aspect, a method includes: receiving first sensor data representing a concentration of particles in an environment having a diameter less than a threshold diameter; receiving second sensor data representing a temperature of the environment; receiving third sensor data representing a humidity of the environment; estimating, based on the first sensor data, second sensor data, and the third sensor data, a concentration of airborne glycol in the environment; and presenting, using the display device, a visual indication representing the concentration of the airborne glycol in the environment.

Implementations of this aspect can include one or more of the following features.

In some implementations, the airborne glycol can include triethylene glycol (TEG) and propylene glycol (PG).

In some implementations, the airborne glycol can include: vaporized TEG, aerosolized TEG, vaporized PG, and aerosolized PG.

In some implementations, the threshold diameter can be 10 μm.

In some implementations, the threshold diameter can be 2.5 μm.

In some implementations, presenting the visual indication representing the concentration of the airborne glycol in the environment can include at least one of: presenting a first visual indication responsive to determining that the concentration of the airborne glycol in the environment is less than a first threshold concentration, presenting a second visual indication responsive to determining that the concentration of the airborne glycol in the environment is greater than or equal to the first threshold concentration and less than a second threshold concentration, and presenting a third visual indication responsive to determining that the concentration of the airborne glycol in the environment is greater than or equal to a third threshold concentration.

In some implementations, the first threshold concentration can be 0.4 mg/m³ and the second threshold concentration can be 9.0 mg/m³.

In some implementations, estimating the concentration of the airborne glycol in the environment can include: multiplying the first sensor data by one or more calibration factors to obtain first calibrated sensor data, and estimating the concentration of the airborne glycol in the environment based on the first calibrated sensor data.

In some implementations, the one or more calibration factors can represent a difference between an output of the first sensor and an output of a reference sensor configured to detect a concentration of particles in a reference environment having a diameter less than the threshold diameter.

In some implementations, the one or more calibration factors can include: a first calibration factor having a value of 0.23 or 0.4, and a second calibration factor having a value of 0.00075.

In some implementations, estimating the concentration of the airborne glycol in the environment can include: determining an adjustment value based on the second sensor data and the third sensor data, multiplying the adjustment value by an additional calibration factor to obtain a calibrated adjustment value, determining a sum of the first calibrated sensor data and the calibrated adjustment value, and estimating the concentration of the airborne glycol in the environment based on the sum of the first calibrated sensor data and the calibrated adjustment value.

In some implementations, estimating the concentration of the airborne glycol in the environment can include determining that the sum of the first calibrated sensor data and the calibrated adjustment value is the concentration of the airborne glycol in the environment.

In some implementations, the airborne glycol can include an aerosolized portion of the glycol, and a vaporized portion of the glycol. The first calibrated sensor data can represent a concentration of the aerosolized portion of the glycol in the environment. The calibrated adjustment value can represent a concentration of the vaporized portion of the glycol in the environment.

In some implementations, the additional calibration factor can be 0.50 mg/m³.

In some implementations, the additional calibration factor can be in a range from 0.4 mg/m³ to 1.0 mg/m³.

In some implementations, the additional calibration factor can be in a range from 0.5 mg/m³ to 0.7 mg/m³.

In some implementations, the adjustment value can increase with an increase in the temperature of the environment.

In some implementations, the adjustment value can decrease with an increase in the humidity of the environment.

In some implementations, the method can also include: receiving a user input comprising instructions to tare at least one of the first sensor data, the second sensor data, or the third sensor data, and responsive too receiving the user input, taring at least one of the first sensor data, the second sensor data, or the third sensor data.

In some implementations, the method can further include transmitting, to a communications network, at least one of: the first sensor data, the second sensor data, the third sensor data, or data representing the concentration of the airborne glycol in the environment.

In some implementations, at least one of the first sensor data, the second sensor data, the third sensor data, or the data representing the concentration of the airborne glycol in the environment can be transmitted to the communications network wirelessly using a wireless transmitter.

In some implementations, at least one of the first sensor data, the second sensor data, the third sensor data, or the data representing the concentration of the airborne glycol in the environment can be transmitted to a glycol dispersal system via the communications network, Further, the method can include regulating, using the glycol dispersal system, the concentration of the airborne glycol in the environment based on at least one of the first sensor data, the second sensor data, the third sensor data, or the data representing the concentration of the airborne glycol in the environment.

In some implementations, the airborne glycol is not visible to a human eye.

The details of one or more embodiments are set forth in the accompanying drawings and the description below. Other features and advantages will be apparent from the description and drawings, and from the claims.

### DESCRIPTION OF DRAWINGS

FIG. 1 is a diagram of an example system for sanitizing an environment using airborne glycol.

FIG. 2 is a diagram of an example glycol detection system.

FIGS. 3A-3D are diagrams of an exterior of an example glycol detection system

FIGS. 4A-4D are diagrams of an interior of an example glycol detection system

FIGS. 5A-5C show results of studies that were performed to identify example calibration factors and/or adjustment values for estimating a concentration of airborne glycol in an environment from aerosolized particle readings.

FIG. 6 shows plots of the saturation of triethylene glycol vapor at various relative humidities and temperatures.

FIG. 7 is a flow chart diagram of an example process for detecting a presence of and/or a concentration of airborne glycol in an environment.

FIG. 8 is a diagram of an example computer system.

Like reference symbols in the various drawings indicate like elements.

### DETAILED DESCRIPTION

### General Overview

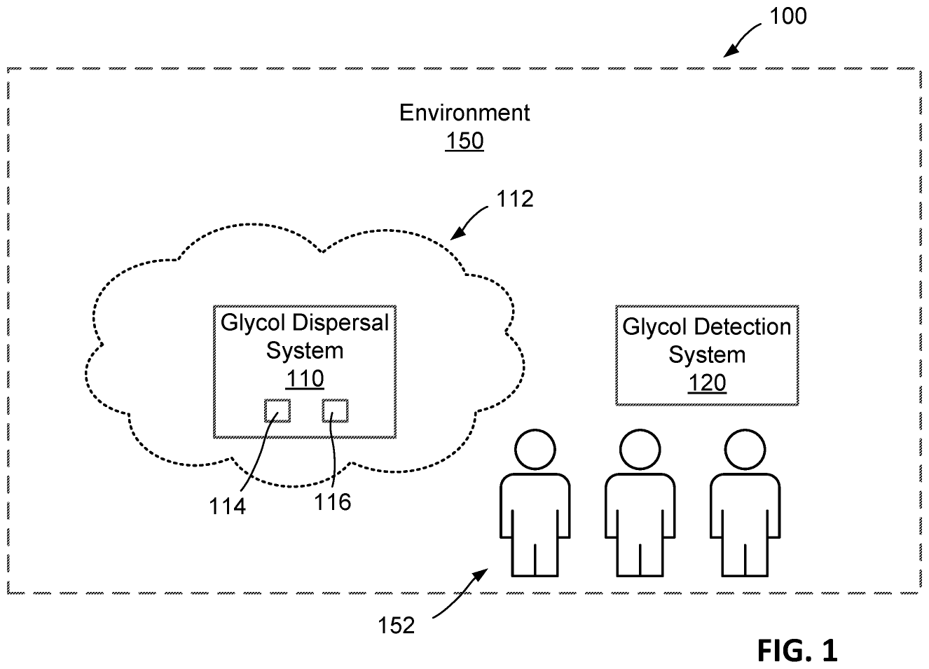

FIG. 1 shows an example system 100 for sanitizing an environment 150 using airborne glycol. The system 100 includes a glycol dispersal system 110 for dispersing airborne glycol into the environment 150, and a glycol detection system 120 for detecting the presence of and/or a concentration of airborne glycol 112 within the environment 150. In some implementations, the system 100 can be configured to neutralize airborne microbes and/or viruses in the environment 150 using airborne glycol, and to measure and regulate the concentration of the airborne glycol in the environment 150 such that the concentration is maintained at a safe and efficacious level.

In some implementations, the environment 150 can be an indoor area, such an area within a permanent or temporary structure. Example indoor areas include rooms, hallways, stairwells, auditoriums, passenger compartments of vehicles, interiors of tents, etc.

The glycol dispersal system 110 is configured to disperse airborne glycol 112 into the environment 150. As shown in FIG. 1, the glycol dispersal system 110 includes a reservoir 114 (e.g., a liquid storage vessel) storing a sanitizing formulation containing glycol as an active ingredient. Further, the glycol dispersal system 110 can include a mechanism 116 for aerosolizing and/or vaporizing the sanitizing formulation into the environment 150. As an example, the mechanism 116 can receive the sanitizing formulation from the reservoir 114, vaporize and/or aerosolize the sanitizing formulation, and direct the aerosolized and/or vaporized sanitizing formulation into the environment 150. In some implementations, the mechanism 116 can include one or more humidifiers, fog/haze machines, smoke generators, impellers, diffusers, ultrasonic agitators, ionizers, nebulizers, atomizers, electro-sprayers, and/or any other components configured to generate vapor and/or aerosols from a liquid.

In some implementations, the sanitizing formulation can include an aqueous solution having triethylene glycol (TEG), propylene glycol (PG), or a combination thereof as active ingredients.

TEG is miscible with water, has a boiling point of 286.5° C. at a pressure of 101.325 kPa, and has a relative low vapor pressure compared to water. Without wishing to be bound by theory, it is believed that TEG is highly hygroscopic and inactivates microbes such as SARS-CoV-2, fungi, Gram Positive and Gram Negative bacteria, and enveloped and non-enveloped viruses, among other microbes, by condensing on microbe-containing particles, droplets, or surfaces until the concentration of TEG becomes sufficiently high to desiccate the pathogen. In addition, without wishing to be bound by theory, it is believed that TEG has very low acute or chronic toxicity when inhaled or ingested (especially at the level used in the air to disinfect an indoor space) and therefore is safe to use in indoor spaces.

In general, the amount of TEG in the sanitizing formulation described herein is not particular limited and can vary as desired. For example, a sanitizing formulation containing a relatively low amount of TEG can achieve the same disinfection effect as a sanitizing formulation containing a relatively high amount of TEG by dispersing the former sanitizing formulation in an indoor space at a higher frequency or in a higher amount. In some implementations, the sanitizing formulation described herein can include TEG in an amount of from at least about 1% (e.g., at least about 5%, at least about 10%, at least about 20%, at least about 30%, at least about 40%, at least about 50%, at least about 52%, at least about 55%, at least about 60%, at least about 65%, at least about 70%, at least about 75%, or at least about 80%) by weight to at most about 99.5% (e.g., at most about 99%, at most about 95%, at most about 90%, at most about 85%, at most about 80%, at most about 75%, at most about 70%, at most about 65%, at most about 60%, or at most about 50%) by weight of the sanitizing formulation. In some implementations, TEG can be 100% of the sanitizing formulation described herein (i.e., without any other ingredient). It is believed that spraying a sanitizing formulation containing a relatively high amount (e.g., at least about 50% by weight) of TEG can increase the efficiency of the disinfection and reduce the frequency of the application of the sanitizing formulation.

In some implementations, the water in the sanitizing formulation described herein can be deionized water. For example, the deionized water can include ions in an amount of from at most about 50 ppm (e.g., at most about 40 ppm, at most about 30 ppm, at most about 20 ppm, at most about 10 ppm, at most about 5 ppm, or at most about 1 ppm) to at least about 1 ppb (e.g., at least about 10 ppb) of the total amount of the deionized water. In some implementations, the water in the sanitizing formulation described herein can be water that has not been deionized.

In some implementations, the sanitizing formulation described herein can include water (e.g., deionized water) in an amount of from at least about 1% (e.g., at least about 5%, at least about 10%, at least about 15%, at least about 20%, at least about 25%, at least about 30%, at least about 35%, at least about 40%, at least about 45%, at least about 48%, at least about 50%, at least about 60%, or at least about 70%) by weight to at most about 99% (e.g., at most about 95%, at most about 90%, at most about 85%, at most about 80%, at most about 75%, at most about 70%, at most about 65%, at most about 60%, at most about 50%, or at most about 48%) by weight of the sanitizing formulation. Without wishing to be bound by theory, it is believed that deionized water can minimize clogging the nozzles (e.g., caused by deposition of minerals in water) of the system used to disperse the sanitizing formulation described herein and therefore can keep the system operating for an extended period of time. Nevertheless, in some implementations, the sanitizing formulation can include water that has not been deionized.

Without wishing to be bound by theory, it is believed that including water in the sanitizing formulation can allow the sanitizing formulation to be readily atomized (e.g., by a humidifier, a fog/haze machine, or a smoke generator) and to form an aerosol in the atmosphere. The aerosol includes fine droplets containing TEG and/or water, which have disinfecting effects and can kill pathogens (e.g., SARS-CoV-2) in the air. In addition, the water in the sanitizing formulation can render the formulation inflammable, thereby resulting in a safer product than TEG alone (which is a flammable liquid having a flash point of 157° C.).

In some implementations, the sanitizing formulation described herein can further include an optional ingredient, such as a glycol different from TEG. In some embodiments, the additional glycol can be a propylene glycol (PG). Without wishing to be bound by theory, it is believed that the additional glycol can either increase the disinfecting effect of the sanitizing formulation or increase the whiteness of the sanitizing formulation (e.g., to indicate that the sanitizing formulation is present in the air). In some embodiments, the sanitizing formulation described herein does not include any additional glycol or any components other than TEG and water.

In some implementations, the sanitizing formulation described herein can include an additional glycol (e.g., a PG) in an amount of from at least about 0.5% (e.g., at least about 1%, at least about 1.5%, at least about 2%, at least about 2.5%, at least about 3%, at least about 3.5%, at least about 4%, at least about 4.5%) by weight to at most about 99% (e.g., at most about 95%, at most about 90%, at most about 80%, at most about 70%, at most about 60%, at most about 50%, at most about 40%, at most about 30%, at most about 20%, at most about 10%, at most about 5%, at most about 4.5%, at most about 4%, at most about 3.5%, at most about 3%, at most about 2.5%, at most about 2%, at most about 1.5%, or at most about 1%) by weight of the formulation.

In some implementations, the sanitizing formulation described herein can include (e.g., comprise, consist essentially of, or consist of) from about 50% to about 90% by weight TEG and from about 10% to about 50% by weight water (e.g., deionized water). In some implementations, the sanitizing formulation described herein can include (e.g., comprise, consist essentially of, or consist of) (1) TEG in an amount of from about 52% to about 90% by weight of the sanitizing formulation; (2) water (e.g., deionized water) in an amount of from about 5% to about 48% by weight of the sanitizing formulation; and (3) PG in an amount of from about 0% to about 5% (e.g., from about 0.5% to about 5%) by weight of the sanitizing formulation. In some implementations, the sanitizing formulation described herein can include (e.g., comprise, consist essentially of, or consist of) about 52.5% by weight TEG, about 1% by weight PG, and about 46.5% by weight water (e.g., deionized water).

As a non-limiting example, the sanitizing formulation can include 52.25% TEG by weight, 1.00% PG by weight, and 46.75% water (e.g., deionized water) by weight. The properties of this particular example of the sanitizing formulation (and pure TEG as a comparative reference) is summarized in the table below

TABLE 1
Properties of an example sanitizing formulation (52.25% TEG by weight,
1.00% PG by weight, and 46.75% water by weight) and TEG.

| | | Example | | | |
|---|---|---|---|---|---|
| | | Sanitizing | | | |
| Test Method | Description | Formulation | | Pure TEG | |
| Visual | Appearance and Odor | Clear/Uniform | | Clear/Uniform | |
| PLTL-15 | Odor Test | No Odor | | No Odor | |
| ASTM D4052 | Specific Gravity @ 20° C. | 1.07-1.09 | | 1.12-1.13 | |
| ASTM D1120 | Boiling Point (° C.) | 103.8° | C. | 285° | C. |
| ASTM D93 | Flash Point (° C.) | Non-flammable | | 157° | C. |
| ASTM D323 | Vapor Pressure (psi) | 0.13 psi @ 25° C. | | 0.0 psi @ 20° C. | |
| ASTM D445 | Viscosity, Kinematic @ 40° C. | 13.96 | cSt | 42.3 | cSt |
| ASTM D117 | Freeze Point ° C. | −29.3° | C. | −7.0° | C. |

The glycol detection system 120 is configured to determine the presence of and/or a concentration of airborne glycol 112 in the environment 150. As an example, the glycol detection system 120 can be configured to determine the presence of and/or a concentration of airborne TEG only, airborne PG only, or a combination thereof. In some implementations, the glycol detection system 120 can be configured to determine a presence and/or a concentration of different forms of airborne glycol, such as vaporized glycol (e.g., vaporized TEG, vaporized PG, etc.) and aerosolized glycol (e.g., aerosolized TEG, aerosolized PG, etc.).

In some implementations, the glycol detection system 120 can present information regarding the detected airborne glycol (e.g., the concentration of airborne glycol) to one or more users 152 within the environment 150, such as using a display device, indicator lights, etc. In some implementations, the glycol detection system 120 can transmit information regarding the detected airborne glycol to one or more remote devices, such as using a wireless or wireless network connection.

In some implementations, the glycol detection system 120 can transmit information regarding the detected airborne glycol to the glycol dispersal system 110 (e.g., to provide feedback to the glycol dispersal system 110 in regulating the dispersal of the sanitizing formulation, such that the airborne glycol is maintained at a safe and efficacious concentration within the environment 150). As an example, if the glycol detection system 120 determines that the concentration of glycol is less than a particular target value, the glycol dispersal system 110 can increase the frequency by which the sanitizing formulation is dispersed into the environment and/or increase the amount of the sanitizing formulation that is dispersed into the environment at a time. As an example, if the glycol detection system 120 determines that the concentration of glycol exceeds a particular target value, the glycol dispersal system 110 can decrease the frequency by which the sanitizing formulation is dispersed into the environment and/or decrease the amount of the sanitizing formulation that is dispersed into the environment at a time.

In some implementations, the glycol detection system 120 can be configured to determine the presence of and/or a concentration of airborne glycol in the environment 150 based, at least in part, on sensor measurements representing (i) a concentration of airborne particulate matter (e.g., liquid droplets) in the environment 150 having a diameter less than a particular threshold diameter (e.g., 2.5 μm, 10 μm, or some other threshold diameter), (ii) a temperature in the environment 150, and (iii) a humidity of the environment 150. For example, the glycol detection system 120 can obtain sensor measurements representing a concentration of airborne particulate matter in the environment 150 having a diameter less than 2.5 μm, calibrate or adjust the sensor measurement based on the temperature and/or humidity of the environment 150, and output the calibrated and/or adjusted sensor measurement as an estimate of the concentration of airborne glycol in the environment 150. Further, in some implementations, the detection system can be configured to determine the presence of and/or a concentration of airborne glycol in an environment without use of a sensor that measures the presence and/or concentration of glycol through chemical processes, such as without the use of an electrochemical sensor or a photoionization sensor.

One or more of the implementations of the glycol detection system 120 can provide various technical benefits. For example, implementations of the glycol detection system 120 can allow users to determine the concentration of airborne glycol in the environment 150 in a quick and efficient manner. This may be particularly useful, for example, in determining whether the concentration of airborne glycol in the environment 150 is sufficiently high to neutralize airborne microbes, fungi and/or viruses within the environment 150, while also sufficiently low to avoid adversely affecting people in the environment 150. Further, implementations of the glycol detection system 120 can be used to detect airborne glycol without the use of chemical processes (e.g., without using electrochemical sensors or a photoionization sensors), which may otherwise increase the complex and/or cost of the glycol detection system 120.

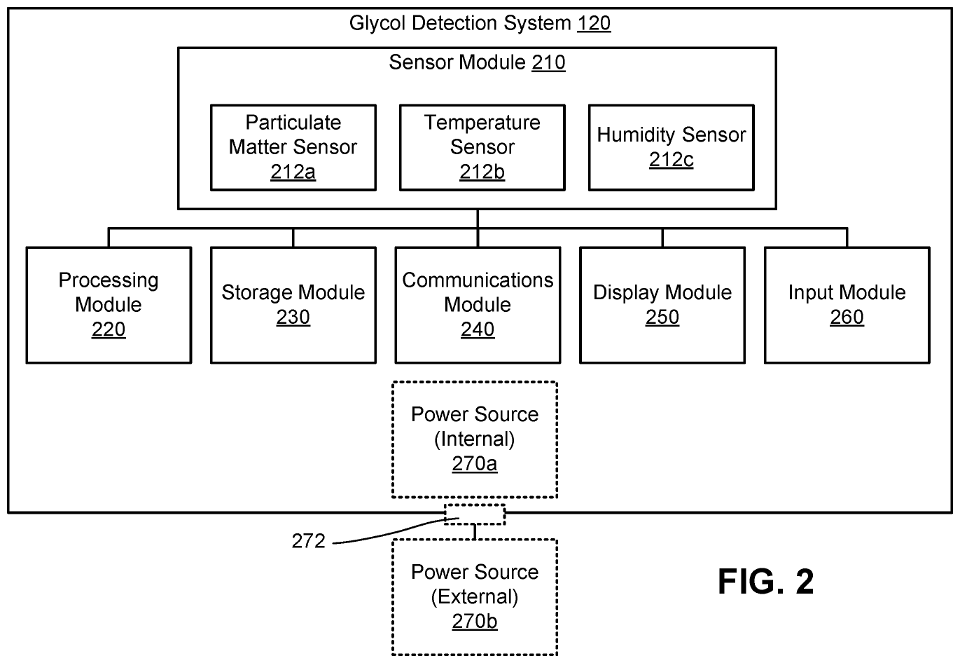

An example of the glycol detection system 120 is shown in greater detail in FIG. 2. As shown in FIG. 2, the glycol detection system 120 includes a sensor module 210, a processing module 220, a storage module 230, a communications module 240, a display module 250, an input module 260, and one or more power sources 270a and/or 270b.

The sensor module 210 is configured to generate sensor measurements representing the environment 150. As an example, the sensor module 210 includes a particulate matter sensor 212a configured to obtain sensor measurement representing a concentration of particulate matter in the environment 150 having a diameter less than a particular threshold diameter. In some implementations, the threshold diameter can be at most 10 μm (e.g., at most 8 μm, at most 6 μm, at most 5 μm, at most 4, at most 2 μm, or at most 1 μm). In these implementations, the sensor measurements may be referred to as Particulate Matter 10 (PM10) measurements. In some implementations, the threshold diameter can be at most 2.5 μm. In these implementations, the sensor measurements may be referred to as Particulate Matter 10 (PM2.5) measurements.

In some implementations, the particulate matter sensor 212a can obtain these measurements by determining the diameters of individual particles, and “binning” particles according to their diameters. For example, the particulate matter sensor 212a can count the number of particles having diameters in a first diameter range (e.g., a first bin), the number of particles having diameters in a second diameter range (e.g., a second bin), the number of particles having diameters in a third diameter range (e.g., a third bin), and so forth. Further, the particulate matter sensor 212a can determine the concentration of particulate matter in the environment 150 having a diameter less than a particular threshold diameter based on one or more of the bins. For instance, the particulate matter sensor 212a can determine the concentration of particulate matter in the environment 150 having a diameter less than 10 μm based on the number of detected particles having a diameter between 0.5 μm and 10 μm.

In some implementations, the particulate matter sensor 212a can include one or more tapered element oscillating microbalances (TEOMs) (which detects particulate matter using a glass tube that vibrates more or less as collected particles accumulate on it), optical photodetectors (which detect particulate matter based on measurements of the light reflected from samples), and/or gravimetric sensors (which detect particulate matter by filtering and weighing samples). In some implementations, the sensor measurements obtained by the particulate matter sensor 212a can represent an approximation of a concentration of aerosolized glycol in the environment.

As another example, the sensor module 210 includes a temperature sensor 212b configured to obtain sensor measurement representing a temperature of the environment 150. As an example, the temperature sensor 212b can include one or more thermometers, thermocouples, thermistors, integrated circuit temperature sensors, etc.

As another example, the sensor module 210 includes a humidity sensor 212c configured to obtain sensor measurement representing a humidity of the environment 150. As an example, the humidity sensor 212c can include one or more hygrometers, such as capacitive hygrometers, resistive hygrometers, thermal hygrometers, gravimetric hygrometers, and/or optical hygrometers.

The processing module 220 processes data stored or otherwise accessible to the glycol detection system 120. For instance, the processing module 220 can retrieve sensor measurements obtained by the sensor module 210, and process the sensor measurements to determine an estimated concentration of airborne glycol in the environment 150. In some implementations, the processing module 220 can store data using the storage module 230. In some implementations, the processing module 220 can transmit data to other systems using the communications module 240. In some implementations, the processing module 220 can present data to one or more users using the display module 250. Example processes that can be performed by the processing module 220 are described in greater detail below.

The storage module 230 maintains information related to the operation of the glycol detection system 120. As examples, the storage module 230 can store sensor measurements obtained by the sensor module 210 and processed sensor measurements generated by the processing module 220. As another example, the storage module 230 can store rules and/or algorithms for processing the sensor measurements obtained by the sensor module 210 (e.g., rules and/or algorithms that are applied to the sensor measurements to determine an estimated concentration of airborne glycol in the environment 150). Although different examples of information are described above, these are merely illustrative. In practice, the storage module 230 can store any information related to the glycol detection system 120.

The communications module 240 allows for the transmission of data to and from the glycol detection system 120. For example, the communications module 240 can be communicatively connected to a communications network, such that it can transmit data to other devices (e.g., the glycol dispersal system 110 and/or any other electronic device), and receive data from other devices via the communication network. As an example, sensor measurements obtained by the sensor module 210 and/or processed by the processing module 220 can be transmitted to the glycol dispersal system 110 (e.g., to provide feedback to the glycol dispersal system 110 in regulating the dispersal of the sanitizing formulation, such that the airborne glycol is maintained at a safe and efficacious concentration within the environment 150). As another example, sensor data obtained by the sensor module 210 and/or processed by the processing module 220 can be transmitted to one or more user devices (e.g., smart phones, tablets, computers, wearable devices, etc.), such that users can monitor the concentration of airborne glycol in the environment 150.

In general, a communications network can be any network through which data can be transferred and shared. For example, a communications network can be a local area network (LAN) or a wide-area network (WAN), such as the Internet. A communications network can be implemented using various networking interfaces, for instance wireless networking interfaces (such as Wi-Fi, Bluetooth, ZigBee, or infrared) or wired networking interfaces (such as Ethernet, serial, or Universal Serial Bus (USB) connection). A communications network also can include combinations of more than one network, and can be implemented using one or more networking interfaces.

The display module 250 is configured to present information to one or more users. As an example, the display module 250 can be configured to present at least a portions of the sensor measurements obtained by the sensor module 210 and/or processed by the processing module 220. For instance, the display module 250 can be configured to present the estimated concentration of airborne glycol in the environment 150. In some implementations, the display module 250 can include one or more display screens, indicator lights, or other display devices for presenting information to a user visually. In addition, in some implementations, the glycol detection system 120 can include other devices for presenting information, such as a speaker (e.g., for presenting information aurally) or a haptic device (e.g., for presenting information in the form of vibrations, pulses, or other haptic stimulations).

The input module 260 is configured to receive inputs by one or more users to control an operation of the glycol detection system 120. As an example, the input module 260 can include one or more touch sensitive surfaces (e.g., touch pad, touch screens, etc.), keypads, buttons, switches, knobs, or other interfaces for receiving user input.

The power sources 270a and 270b provide electrical power to the glycol detection system 120 and each of its constituent components. In some implementations, the glycol detection system 120 can include an internal power source 270a (e.g., a battery, capacitor, etc.). In some implementations, the glycol detection system 120 can be electrically coupled to an external power source 270b (e.g., an external electrical grid, an external power generator, an external battery, etc.) via a connection interface 272 (e.g., a plug, socket, receptacle, port, connector, etc.).

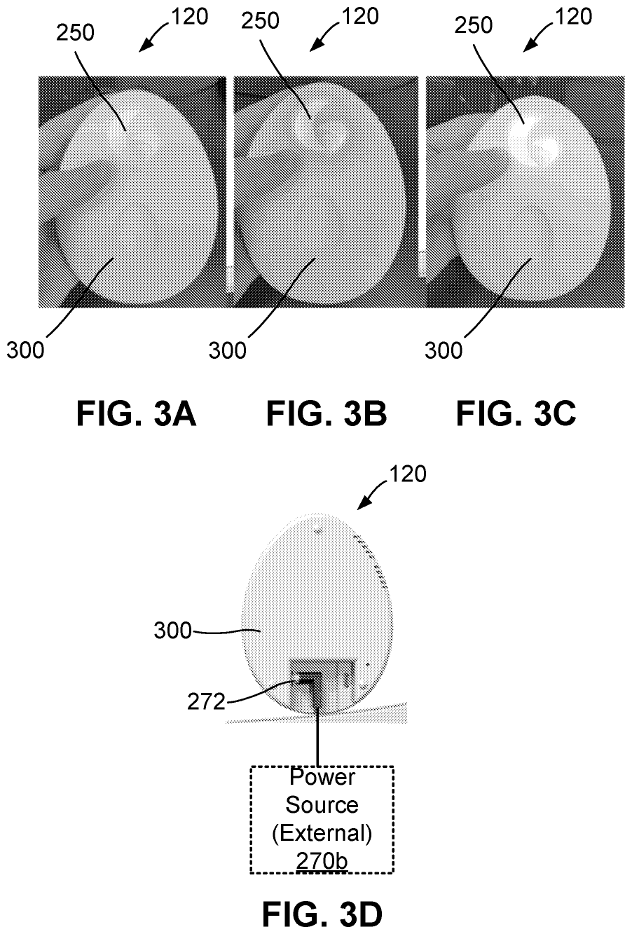

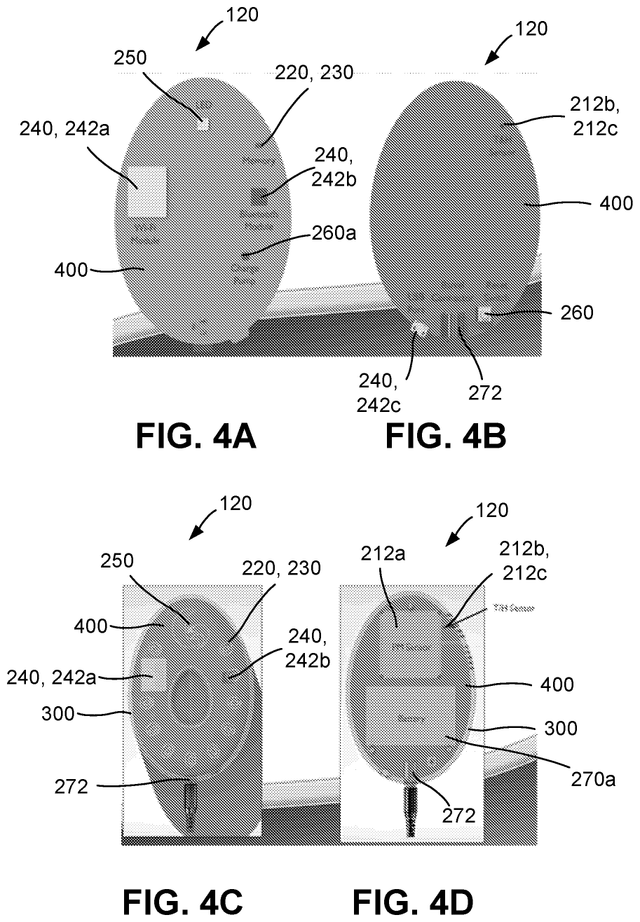

An example physical configuration of the glycol detection system 120 is shown in FIGS. 3A-3C (depicting the front of the glycol detection system 120), FIG. 3D (depicting the rear of the glycol detection system 120), and FIGS. 4A-4D (depicting the internal components of the glycol detection system 120).

As shown in FIGS. 3A-3C, the glycol detection system 120 can include a housing 300 at least partially enclosing some or all of the components of the glycol detection system 120. Further, the glycol detection system 120 can include a display module 250 (e.g., one or more indicator lights) that are visible along an exterior of the housing 300.

In this example, the display module 250 is configured to present a visual indication representing the estimated concentration of airborne glycol in the environment 150. For example, the display module 250 can be configured to (i) emit light having a first color and/or according to a first pattern when the estimated concentration of airborne glycol in the environment 150 is within a first range (e.g., less than a first threshold concentration), (ii) emit light having a second color and/or according to a second pattern when the estimated concentration of airborne glycol in the environment 150 is within a second range (e.g., greater than or equal to the first threshold concentration, and less than a second threshold concentration), and (iii) emit light having a third color and/or according to a third pattern when the estimated concentration of airborne glycol in the environment 150 is within a third range (e.g., greater than the second threshold concentration).

As an example, the display module 250 can be configured to emit white light when the estimated concentration of airborne glycol in the environment 150 is within the first range. Further, the display module 250 can be configured to emit blue light when the estimated concentration of airborne glycol in the environment 150 is within the second range. Further, the display module 250 can be configured to emit yellow light when the estimated concentration of airborne glycol in the environment 150 is within the third range. Other colors and/or patterns (e.g., blinking, pulsing, solid, etc.) are also possible, depending on the implementation.

In some implementations, the ranges can correspond to the efficacy and/or safety of airborne glycol at certain concentrations in an environment. For example, the second range can correspond to concentrations of airborne glycol that are suitable for effectively neutralizing airborne microbes, fungi and/or viruses in the environment, without adversely affecting people in the environment. As another example, the first range can correspond to concentrations of airborne glycol that are not sufficiently high to effectively neutralize airborne microbe and/or viruses in the environment. As another example, the third range can correspond to concentrations of airborne glycol that exceed certain specified safety limits regarding extended exposure to airborne glycol. As another example, the third range can correspond to concentrations of airborne glycol that are higher than required for efficacy but do not exceed certain specified safety limits regarding extended exposure to airborne glycol.

In some implementations, the first range can be less than 0.5 mg/m³, the second range can be greater than or equal to 0.5 mg/m³ and less than 9.0 mg/m³, and the third range can be greater than or equal to 9.0 mg/m³. In some implementations, these ranges can refer to a combination of both aerosolized and vaporized forms of glycol.

In some implementations, the first range can be less than 0.04 mg/m³, the second range can be greater than or equal to 0.02 mg/m³ and less than 9.0 mg/m³, and the third range can be greater than or equal to 9.0 mg/m³. In some implementations, these ranges can refer to aerosolized forms of glycol only (e.g., and not vaporized forms of glycol).

In some implementations, the first range can be less than 0.4 mg/m³, the second range can be greater than or equal to 0.4 mg/m³ and less than 9.0 mg/m³, and the third range can be greater than or equal to 9.0 mg/m³. In some implementations, these ranges can refer to a combination of both aerosolized and vaporized forms of glycol.

Other ranges are also possible, depending on the implementation.

In some implementations, at least part of the second range can correspond to concentrations of airborne glycol that are suitable for effectively neutralizing airborne microbes and/or viruses in the environment, without adversely affecting people in the environment, and without being visible in the air to the human eye. This can be beneficial, for example, as certain concentrations of airborne glycol may be sufficiently high to neutralize airborne microbes and/or viruses in the environment, while remaining “invisible” to people in the environment (e.g., to improve the aesthetic quality of the environment, avoid startling or distracting people in the environment, etc.). For example, the second range can include, at least in part, concentrations of airborne glycol from 0.04 mg/m³ to 0.1 mg/m³.

As shown in FIG. 3D the glycol detection system 120 can include a connection interface 272 (e.g., a plug, socket, receptacle, port connector, etc.) along an exterior of the housing 300 for electrically coupling the glycol detection system 120 to an external power source 270b.

As shown in FIGS. 4A-4D, the glycol detection system 120 can include one or more electronic components mounted to one or more printed circuit boards (PCBs) 400. In this example, the glycol detection system 120 includes sensor module including a particulate matter sensor 212a, a temperature sensor 212b, and a humidity sensor 212c mounted to the PCB 400. Further, the glycol detection system 120 includes a processing module 220 including one or more processors mounted to the PCB 400. The glycol detection system 120 also includes a storage module 230 include one or more memory devices (e.g., memory chips) mounted to the PCB 400. The glycol detection system 120 also includes a communications module 240 including a Wi-Fi transceiver 242a, a Bluetooth transceiver 242b, and a wired communications interface 242c (e.g., a universal serial bus (USB) port) mounted to the PCB 400. The glycol detection system 120 also includes a display module 250 including one or more indicator lights (e.g., light emitting diodes) mounted to the PCB 400. The glycol detection system 120 also includes an internal power source 270a (e.g., a battery and associated charge pump), and a connection interface 272 for electrically coupling the glycol detection system 120 to an external power source 270b (not shown in FIGS. 4A-4D) mounted to the PCB 400. The glycol detection system 120 also includes an input module 260 (e.g., one or more buttons or switches that enable a user to input commands to the glycol detection system 120) mounted to the PCB 400.

In some implementations, the glycol detection system 120 can be configured to estimate the concentration of airborne glycol on a continuous basis. In some implementations, the glycol detection system 120 can be configured to estimate the concentration of airborne glycol in a periodic basis (e.g., every second, 10 seconds, 30 seconds, 1 minute, 5 minutes, or according to any other period). In some implementations, the glycol detection system 120 can be configured to estimate the concentration of airborne glycol at a single point in time (e.g., in response to a command from a user).

In some implementations, the glycol detection system 120 can generate multiple estimates of the concentration of airborne glycol in a sequence, and determine an average (e.g., a mean) of two or more of the estimates. Further, the glycol detection system 120 can present the average of the estimates to a user (e.g., via the display module 250), and/or transmit the average of the estimates to another device (e.g., via the communications module 240). Averaging the estimates can be beneficial, for example, in reducing sudden fluctuations in the output of the glycol detection system 120 (which may be more difficult for a user to interpret).

As an example, when the glycol detection system 120 is initially activated, the glycol detection system 120 can estimate the concentration of airborne glycol continuously, and determine an average of the estimates every minute (e.g., in successive one minute windows). During this time, the glycol detection system 120 can indicate that it is in the process of obtaining sensor measurements. For instance, the glycol detection system 120 can use the display module 250 to emit blue light according to a slow pulsing pattern.

If the average of the estimates is within a target concentration range (e.g., the second concentration range) for three consecutive averages (e.g., over a three minute time span), the glycol detection system 120 can switch to obtaining an average of the estimates every five minutes (e.g., in successive five minute windows). Further, the glycol detection system 120 can indicate that the estimate concentration of airborne glycol is in the target range. For instance, the glycol detection system 120 can use the display module 250 to emit blue light according to a solid non-pulsing pattern.

If subsequently the average of the estimates is no longer within the target concentration range (e.g., over a five minute timespan), the glycol detection system 120 can again determine an average of the estimates every minute (e.g., in successive one minute windows). During this time, the glycol detection system 120 can indicate that the concentration of airborne glycol is no longer in the target concentration range. For example, if the average of the estimates is less than the target concentration range, the glycol detection system 120 can use the display module 250 to emit blue light according to a slow pulsing pattern. As another example, if the average of the estimates is greater than the target concentration range, the glycol detection system 120 can use the display module 250 to emit yellow light.

Other colors and/or patterns (e.g., blinking, pulsing, solid, etc.) are also possible, depending on the implementation.

In some implementations, the glycol detection system 120 can be configured to obtain measurements from the particulate matter sensor 212a on a continuous basis (e.g., by continuously counting the number of particles having diameters less than a threshold value, as a representation of the concentration of those particles in the air). Further, the glycol detection system 120 can determine when the measurements achieve a steady state over a rolling time window (e.g., the variation of the measurements does not exceed ±20% over a three minute rolling time window). Upon determining that the measurements achieved a steady state over the rolling time window, the glycol detection system 120 can report the estimated concentration of airborne glycol to a user (e.g., using the indicator lights described herein). However, if the measurement have not yet achieved a steady state over the rolling time window, the glycol detection system 120 can refrain from reporting any updates to the estimated concentration of airborne glycol to a user (e.g., by refraining from changing the status of the indicator lights). Further, in some implementations, the glycol detection system 120 can report a rolling average of the estimated concentration of airborne glycol over time.

This technique can be useful, for example, in presenting more accurate measurements to the user and/or presenting measurements that are subject to a lesser degree of variability. For instance, in at least some implementations, estimates by the glycol detection system 120 regarding the concentration of airborne glycol may be less inaccurate when the airborne glycol has not achieved vapor saturation in the environment. Further, the time at which the airborne glycol has achieved vapor saturation in the environment can be determined by identifying the time at which the count of the number of particles having diameters less than a threshold value reaches a steady state. Accordingly, by selectively reporting estimates when the counted number of particles having diameters less than a threshold value reaches a steady state, the output of the glycol detection system 120 is more accurate and/or reliable.

As described above, in some implementations, the steady state of a measurement can be determined based on a steady state condition in the count of the number of particles having diameters less than a threshold value (e.g., a steady state condition in PM10 and/or PM2.5 measurements). However, in some implementations, the steady state of a measurement can be determined based on a steady state condition in the calculated mass concentration of particles. As an example, the mass concentration of particles can be calculated using Eq. 1 described below, where E is set to 1.

In some implementations, the glycol detection system 120 can be configured to obtain measurements at a pre-determined time after it is turned on and/or activated. As an example, the glycol detection system 120 can be configured to obtain measurements 30 seconds after it is turned on and/or activated.

In some implementations, the glycol detection system 120 can be configured to perform a taring operation with respect to the sensor measurements obtained by the sensor module 210. As an example, prior to the dispersal of glycol into the environment by the glycol dispersal system 110, the glycol detection system 120 can obtain a baseline measurement of the concentration of airborne particulate matter in the environment having a diameter less than a particular threshold diameter (e.g., 10 μm). Further, subsequent to the dispersal of glycol into the environment by the glycol dispersal system 110, the glycol detection system 120 can obtain one or more additional measurements of the concentration of airborne particulate matter in the environment having a diameter less than the particular threshold diameter. Further, the glycol detection system 120 can subtract the baseline measurement from each of the additional measurements (e.g., to tare each of the additional measurements by the baseline measurement). This can be particularly beneficial, for example, in improving the accuracy of the output of the glycol detection system 120 in environments having a significant amount of dust or other particulate matter.

In some implementations, the glycol detection system 120 can determine whether a taring operation should be performed based on sensor measurements obtained by the sensor module 210. As an example, prior to the dispersal of glycol into the environment by the glycol dispersal system 110, the glycol detection system 120 can obtain a baseline measurement of the concentration of airborne particulate matter in the environment having a diameter less than a particular threshold diameter (e.g., 10 μm). If the measurements are above a particular threshold level (e.g., indicating the presence of a significant amount of particulate matter that is not airborne glycol), the glycol detection system 120 can determine that a taring operation should be performed to improve the accuracy of subsequent measurements.

In some implementations, the glycol detection system 120 can indicate to a user that a taring operation should be performed (e.g., using the display module 250), and a user can manually instruct the glycol detection system 120 to perform a taring operation. In some implementations, the glycol detection system 120 can perform the taring operation automatically.

As described above, the glycol detection system 120 can be configured to estimate concentration of airborne glycol in the environment based, at least in part, on sensor measurements representing (i) a concentration of airborne particulate matter in the environment having a diameter less than a particular threshold diameter (e.g., 10 μm), (ii) a temperature in the environment, and (iii) a humidity of the environment. In some implementations, the glycol detection system 120 can be configured to generate sensor output X (representing an estimated concentration of airborne glycol in the environment) according to the following equation:

$$X = ( A * B * C ) + ( D * E ) . \qquad ( Eq . 1 )$$

A is the concentration of airborne particulate matter in the environment having a diameter less than 10 μm (PM10). In some implementations, A can be the concentration of airborne particulate matter in the environment having a diameter between 0.5 μm to 10 μm. Further, B, C, D, and E are adjustment values and/or calibration factors. At least some of B, C, D, and/or E can be determined based on the temperature of the environment and/or the humidity of the environment.

In some implementations, the calibration factor B can represent a correlation between sensor measurements obtained by the particulate matter sensor 212a and sensor measurements obtained by a reference sensor (e.g., a calibrated sensor meeting certain accuracy and/or precision specifications) under specific reference conditions.

As an example, the particulate matter sensor 212a can be an uncalibrated sensor (e.g., a particulate matter sensor such as the Sensirion SPS30PM sensor, produced by Sensirion AG, Stafa, Switzerland). Further, the reference sensor can be a calibrated sensor (e.g., a particulate matter sensor TSI AM520, produced by TSI Inc., Shoreview, Minnesota) calibrated to ISO Standard 12103-1 A1 using “Ultrafine Arizona Road Dust” (as specified in ISO Standard 12103-1 A1). “Ultrafine Arizona Road Dust” has a density of 2.7 g/cm³, whereas at least some implementations of the sanitizing formulation have a density of 1.08 g/cm³. Accordingly, in at least some implementations, the calibration factor B can be 1.08/2.7=0.4.

In some implementations, the calibration factor C can represent an additional correlation between sensor measurements obtained by the particulate matter sensor 212a and sensor measurements obtained by a reference sensor (e.g., a calibrated sensor meeting certain accuracy and/or precision specifications) under specific reference conditions.

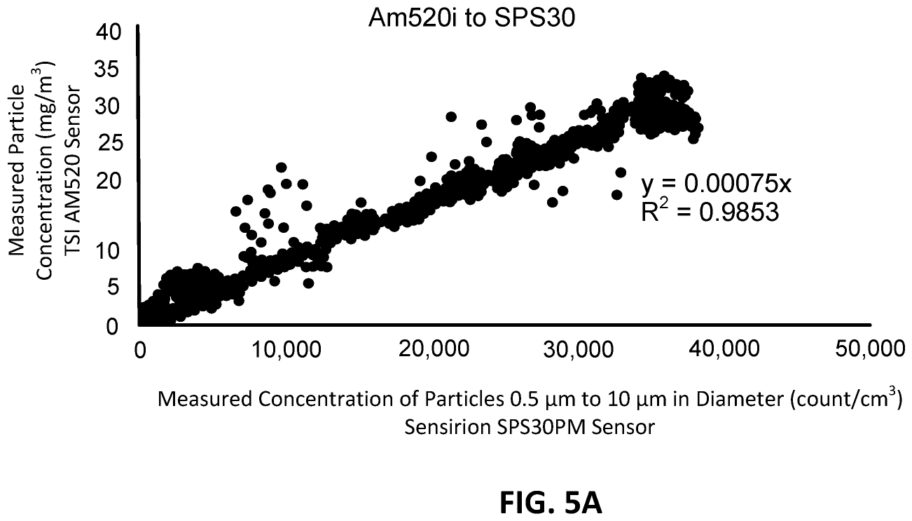

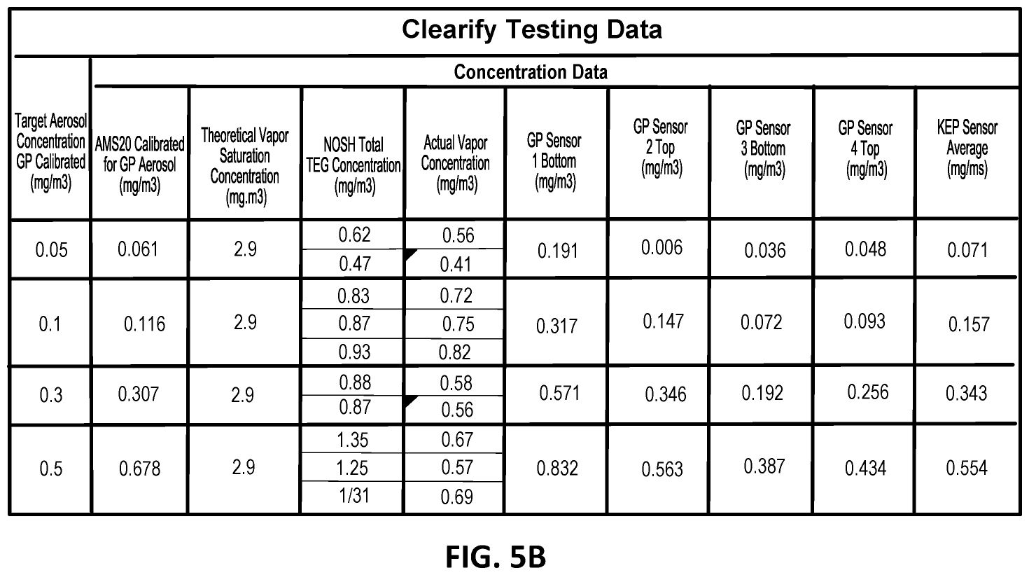

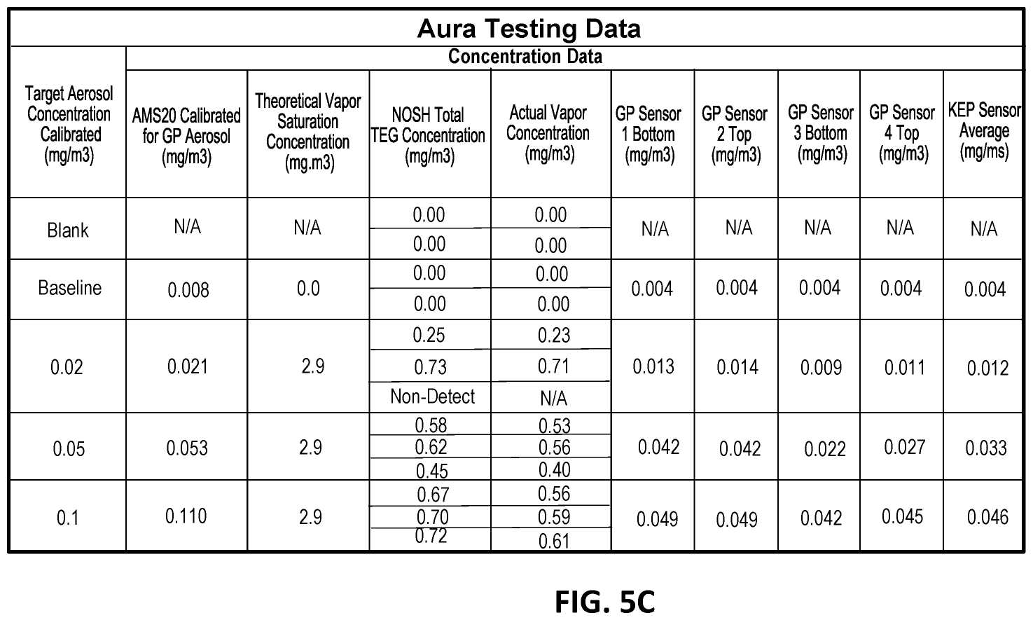

For example, FIG. 5A shows a scatter plot comparing measurements obtained by the Sensirion SPS30PM sensor (horizontal axis) and measurements obtained by the TSI AM520 sensor (vertical axis). After removing outliers, the measurements were found to have a linear correlation with a slope of 0.00075. Accordingly, in at least some implementations, the calibration factor C can be 0.00075.

In some implementations, the adjustment value D can represent at least some of the vaporized glycol (e.g., vaporized TEG and/or vaporized PG) that cannot be directly detected by a particulate matter sensor (as vapor does not have any particles that can be directly detected by a particulate matter sensor). The adjustment value factor D can be determined empirically through experimental testing. In some implementations, the adjustment value D can be expressed in units of mg/m³.

As an example, tests were performed to determine the amount of vaporized glycol that is non-detectable by a particulate matter sensor (Sensirion SPS30PM sensor). The results are summarized in the tables shown in FIGS. 5B and 5C. Columns titled “Actual Vapor Concentration (mg/m³) shows the constant factors using two different technologies: cold dispersion (nebulization) and heated dispersion (vaporization).

As shown in FIGS. 5B and 5C, the average of the Actual Vapor Concentration (mg/m³) for various ranges between 0.06 mg/m³ and 0.6 mg/m³ that was found in addition to the SPS30 measured value was 0.59 mg/m³. To correct the sensor output to account for this factor, an additional value is added to the corrected and calibrated sensor output. In some implementations, this additional value can be selected from the range 0.4 mg/m³ to 1.0 mg/m³. In some implementations, this additional value can be selected from the range 0.5 mg/m³ to 0.7 mg/m³.

Further, additional testing was performed using a collection device (XAD-7 OSHA Versatile Sampler (OVS) tube) that collects, in a controlled manner, both vaporized and aerosolized glycol (e.g., vaporized TEG, aerosolized TEG, vaporized PG, aerosolized PG, etc.). In particular, the total amount of airborne glycol in a testing environment (including both vaporized and aerosolized glycol) was measured using the OVS tube via gas chromatography (e.g., using a flame ionization technique). Further, the measured amount was used to calibrate and/or validate the measurements determined using the glycol detection system 120 (e.g., to improve the accuracy of the measurements based on using the glycol detection system 120). As shown in FIGS. 5B and 5C, the additional calibration factor being in a consistent range between 0.4 mg/m³ and 0.7 mg/m³. At higher concentrations in the air, higher linear increased vaporized and aerosolized glycol is present.

In some implementations, the adjustment value D can be a constant value. For example, the adjustment value D can be 0.50 mg/m³.

In some implementations, the adjustment value D can vary depending on the PM10 particle count by the sensor. For example, if the sensor detects a PM10 particle count in a first range, the adjustment value D can be a first value. Further, if the sensor detects a PM10 particle count in a second range, the adjustment value D can be a second value. Further still, if the sensor detects a PM10 particle count in a third range, the adjustment value D can be a third value, and so forth. This can be beneficial, for example, in enabling the final output of the sensor to be more accurately calibrated across a range of operating conditions.

In some implementations, if the sensor detects a PM10 particle count in a first range from 100 to 134, the adjustment value D can be a 0.4 mg/m³. Further, if the sensor detects a PM10 particle count in a second range from 134-2499, the adjustment value D can be 0.5 mg/m³. Further still, if the sensor detects a PM10 particle count in a third range greater than or equal to 2500, the adjustment value D can be a 1.0 mg/m³. This set of ranges and adjustment values has been found to generate a particularly accurate final output of the sensor in at least some implementations.

In some implementations, if the sensor detects a PM10 particle count in a first range from 100 to 134, the adjustment value D can be a 0.3 mg/m³. Further, if the sensor detects a PM10 particle count in a second range from 134-2499, the adjustment value D can be 0.4 mg/m³. Further still, if the sensor detects a PM10 particle count in a third range greater than or equal to 2500, the adjustment value D can be a 1.0 mg/m³. This set of ranges and adjustment values has also been found to generate a particularly accurate final output of the sensor in at least some implementations.

Nevertheless, depending on the implementation, other sets of ranges and adjustments values can be used.

In some implementations, the adjustment value D can vary depending on the PM2.5 particle count by the sensor. For example, if the sensor detects a PM2.5 particle count in a first range, the adjustment value D can be a first value. Further, if the sensor detects a PM2.5 particle count in a second range, the adjustment value D can be a second value. Further still, if the sensor detects a PM2.5 particle count in a third range, the adjustment value D can be a third value, and so forth. This can be beneficial, for example, in enabling the final output of the sensor to be more accurately calibrated across a range of operating conditions.

In some implementations, if the sensor detects a PM2.5 particle count in a first range from 100 to 134, the adjustment value D can be a 0.4 mg/m³. Further, if the sensor detects a PM2.5 particle count in a second range from 134-2499, the adjustment value D can be 0.5 mg/m³. Further still, if the sensor detects a PM2.5 particle count in a third range greater than or equal to 2500, the adjustment value D can be a 1.0 mg/m³. This set of ranges and adjustment values has been found to generate a particularly accurate final output of the sensor in at least some implementations.

In some implementations, if the sensor detects a PM2.5 particle count in a first range from 100 to 134, the adjustment value D can be a 0.3 mg/m³. Further, if the sensor detects a PM2.5 particle count in a second range from 134-2499, the adjustment value D can be 0.4 mg/m³. Further still, if the sensor detects a PM2.5 particle count in a third range greater than or equal to 2500, the adjustment value D can be a 1.0 mg/m³. This set of ranges and adjustment values has also been found to generate a particularly accurate final output of the sensor in at least some implementations.

Nevertheless, depending on the implementation, other sets of ranges and adjustments values can be used.

In some implementations, the calibration value E can represent variations in the amount of vaporized glycol in an environment based on temperature and/or humidity.

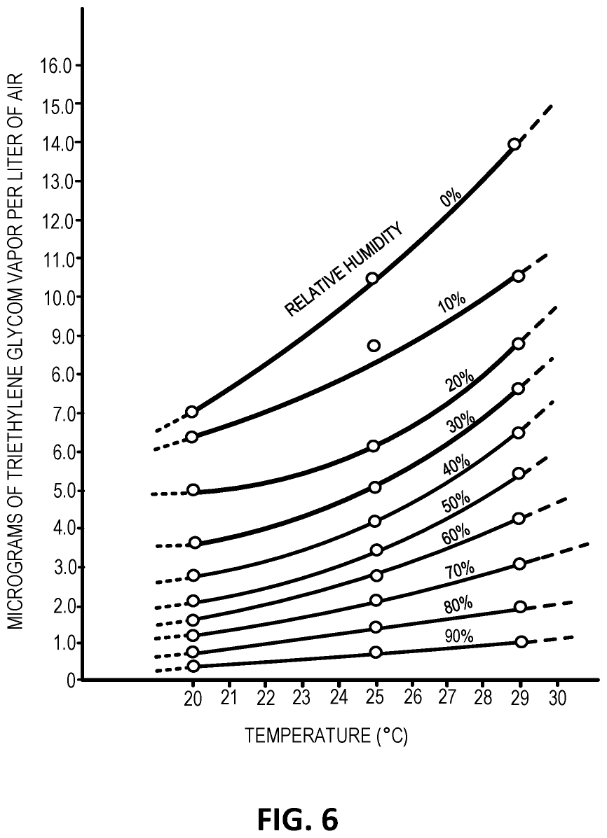

For example, FIG. 6 shows plots of the saturation of TEG vapor at various relative humidities for temperature from 20.0° C. to 29.0° C., according to theoretical analyses. As shown in FIG. 6, vapor is affected by both temperature and humidity. In particular, higher temperature allows for more vapor to be supported in the air and a higher humidity lowers the available space for vaporized glycol. However, at the levels of airborne glycol that are effective in neutralized airborne microbes and viruses (e.g., between 0.4 mg/m³ and 9.0 mg/m³), operationally the vapor constant is lower than any of the theoretical vapor maximums in the graph curved shown in FIG. 6.

As shown in FIG. 6, higher humidity prohibits a vapor saturation close to the range used for efficacy. In at least some use cases, the system 100 is operated in an indoor area having conditioned air. Accordingly, a temperature range of 20° C. to 24° C. and a relative humidity of 30% may be representative of the environment. However, accounting for environmental conditions that may occur, at 90% and greater humidity, a safety factor of 0.7 can be multiplied with the output. Further, between 80% and 90% humidity, a 0.9 factor can be multiplied with the output. This accounts for the additional aerosolized glycol in the air to account for the environmental conditions that require a higher aerosol content to maintain with the efficacy guidelines.

In at least some implementations, if (i) the measured humidity is 90% or greater and (ii) the measured temperature is less than 30° C., the calibration factor E can be 0.07. Otherwise, if (i) the measured humidity is greater than 80% and (ii) the measured temperature is less than 25° C., the calibration factor E can be 0.15. Otherwise, the calibration factor E can be 0. This can be represented in pseudo-code, as follows:

```
IF ((humidity) >= 90% AND (temperature) < 30°C), E
= 0.07;
  ELSE IF ((humidity) > 80% AND (temperature) <
25°C), E = 0.15;
    ELSE E = 1;
```

Other techniques for calculating E are also possible, depending on the implementation.

Therefore, in at least some implementations, the glycol detection system 120 can be configured to generate sensor output X (representing an estimated concentration of airborne glycol in the environment) according to the following equation:

$$X = ( A * 0.4 * 0.00075 ) + ( 0.50 ) * E , \qquad ( Eq . 2 )$$

where E is determined based on the temperature and humidity measurements obtained by the temperature sensor 212b and the humidity sensor 212c, respectively.

Although example calibration factors are described above, in practice, the calibrations factors may differ depending on the implementation. For example, further testing and/or studies may indicate that a different calibration factor can be used to produce more accurate estimates regarding the amount of vaporized glycol in an environment.

As an example, in some implementations, the calibration factor B can instead be 0.23. The value is based on a determination that, when the average recorded concentration value by the glycol detection system 120 is plotted against the laboratory measured concentrations of the glycol (e.g., including both aerosolized and vaporized forms of glycol), the relationship between the two concentration values can be presented by a linear regression model. In this model, the slope of the curve determines the calibration factor B. In certain experimental studies, the slope was determined be to 0.23.

Thus, in at least some implementations, the glycol detection system 120 can be configured to generate sensor output X (representing an estimated concentration of airborne glycol in the environment) according to the following equation:

$$X = ( A * 0.23 * 0.00075 ) + 0.50 * E . \qquad ( Eq . 3 )$$

In some implementations, the calibration factors described above may be particularly suitable for determining the concentration of airborne TEG alone (e.g., and not airborne PG). Nevertheless, in some implementations, the calibration factors above can be used to determine the concentration of both airborne TEG and airborne PG, or airborne PG alone.

In some implementations, the output of the glycol detection system 120 (e.g., in accordance with the calibration factors described above) may be within a particular tolerance range of the actual concentration of airborne glycol in the environment. As an example, in some implementations, the output of the glycol detection system 120 can be within a tolerance range of ±0.10 mg/m³ of the actual concentration of airborne TEG in the environment (e.g., as measured using NIOSH test equipment). As an example, in some implementations, the output of the glycol detection system 120 can be within a tolerance range of ±0.15 mg/m³ of the actual concentration of airborne TEG in the environment (e.g., as measured using NIOSH test equipment).

Further, in some implementations, the glycol detection system 120 can be configured to generate sensor output X according to only a subset of the calibration factors discussed above. As an example, in some implementations, the glycol detection system 120 can be configured to generate sensor output X according to the following equation:

$$X = ( A * B * C ) + D . \qquad ( Eq . 3 )$$

As another example, in some implementations, the glycol detection system 120 can be configured to generate sensor output X according to the following equation:

$$X = A * B * C . \qquad ( Eq . 4 )$$

### Additional Experimental Studies

Additional experimental studies were conducted to assess and validate the performance of example glycol detection systems 120. A summary of these additional experimental studies is provided below.

### Scope:

The scope of these experimental studies was to assess and validate the performance of an example implementation of the glycol detection system 120 in detecting the concentration of airborne glycol (e.g., including both aerosolized and vaporized forms of glycol).

### Methods:

In these studies, airborne glycol was dispersed into various environments using two different implementations of a glycol dispersal system (e.g., a nebulizing glycol dispersal system and a vaporizing glycol dispersal system). Further, an implementation of a glycol detection system 120 was used to estimate the concentration of airborne glycol in the environments (e.g., including both aerosolized and vaporized forms of glycol).

In these studies, a glycol detection system 120 was used to detect and report the concentration of aerosolized micro-droplets of glycol (e.g., having a diameter between 0.5 μm and 10 μm) in the air. Further, based on the measurements, the glycol detection system 120 was used to estimate the concentration of total airborne glycol (e.g., including both aerosolized forms of glycol, and vaporized forms of glycol that cannot be directly detected by the glycol detection system 120). The estimates were performed using the calibration factors described with respect to Eq. 1 above, using a value of 0.4 as the calibration factor B). Nevertheless, it is believed that similar results may be obtained by using a value of 0.23 as the calibration factor B.

As a point of comparison, NIOSH (Method 5523) test equipment was used to determine the concentration of total airborne glycol (e.g., including both aerosolized forms of glycol and vaporized forms of glycol).

The glycol detection system 120 and NIOSH test equipment were placed in close proximity to one another in each test environment, and were operated concurrently to collect measurements.

Studies were conducted in two different environments: a controlled laboratory environment (laboratory) and a “real-world” office environment.

### Results and Discussion:

The measurements and estimates are summarized in Table 2 below.

TABLE 2
Measured and estimated concentrations of airborne glycol
using a glycol detection system 120 and a NOISH system.

| (A) | | | | | | |
|---|---|---|---|---|---|---|
| Measured | (B) | | | | | |
| Average | Estimated | | | | | |
| Concentration | Concentration | (C) | (D) | | | |
| of Aerosolized | of Total | Measured | Average | | | |
| Glycol over 30 | Airborne | Concentration | Concentration | (E) | | |
| minutes | Glycol | of Total | of Total | Difference | | |
| (mg/m³) | (mg/m3) | Airborne | Airborne | between B | | |
| (Glycol | (Glycol | Glycol | Glycol | and D | (F) | (G) |
| Detection | Detection | (mg/m³) | (mg/m3) | (Absolute | Test | Dispersal |
| System) | System) | (NIOSH) | (NIOSH) | Value) | Location | System |
| 0.005720 | 0.60572 | 0.54 | 0.61 | 0.00 | Lab | Nebulizer |
| | | 0.57 | | | | |
| | | 0.73 | | | | |
| 0.010920 | 0.61092 | 0.25 | 0.49 | 0.12 | Office | Nebulizer |
| | | 0.73 | | | | |
| | | N/A | | | | |
| 0.014040 | 0.61404 | 0.41 | 0.65 | 0.04 | Lab | Nebulizer |
| | | 0.69 | | | | |
| | | 0.85 | | | | |
| 0.027560 | 0.62756 | 0.58 | 0.55 | 0.08 | Office | Nebulizer |
| | | 0.62 | | | | |
| | | 0.45 | | | | |
| 0.029640 | 0.62964 | 0.43 | 0.59 | 0.04 | Lab | Nebulizer |
| | | 0.78 | | | | |
| | | 0.57 | | | | |
| 0.046800 | 0.6468 | 0.57 | 0.73 | 0.08 | Lab | Vaporizer |
| | | 0.89 | | | | |
| | | N/A | | | | |
| 0.057200 | 0.6572 | 0.67 | 0.70 | 0.04 | Office | Nebulizer |
| | | 0.70 | | | | |
| | | 0.72 | | | | |
| 0.083200 | 0.6832 | 0.95 | 0.97 | 0.29 | Lab | Vaporizer |
| | | 1.00 | | | | |
| | | N/A | | | | |

Column (A) represents the measured average concentrations of aerosolized glycol over 30 minutes, as determined by the glycol detection system 120. Further, Column (B) represents an estimated concentration of the total airborne glycol (e.g., including both aerosolized and vaporized forms of glycol) determined based on the measurements obtained by the glycol detection system 120 (e.g., using the calibration factors described herein, such as with reference to Eq. 1).

Column (C) represents the measured concentrations of the total airborne glycol, as determined by the NIOSH system. Further, Column (D) represents the average of these measurements.

Further, Column (E) represents the absolute value of the difference between Column (B) and Column (D) (e.g., representing the difference between (i) the concentration of the total airborne glycol as estimated by the glycol detection system 120, and (ii) the concentration of the total airborne glycol as measured by the NIOSH system).

Column (F) indicates the test location or environment for each study Column (G) indicates the type of dispersal system that was used to disperse the glycol into the test location or environment.

As shown in Table 2, there is close agreement between (i) the concentration of the total airborne glycol as estimated by the glycol detection system 120 and (ii) the concentration of the total airborne glycol as measured by the NIOSH system). For example, there is a direct correlation between the two. Further, the correlation was not affected by the test environment or the type of dispersal system that was used to disperse the glycol into the test location or environment.

### Example Processes

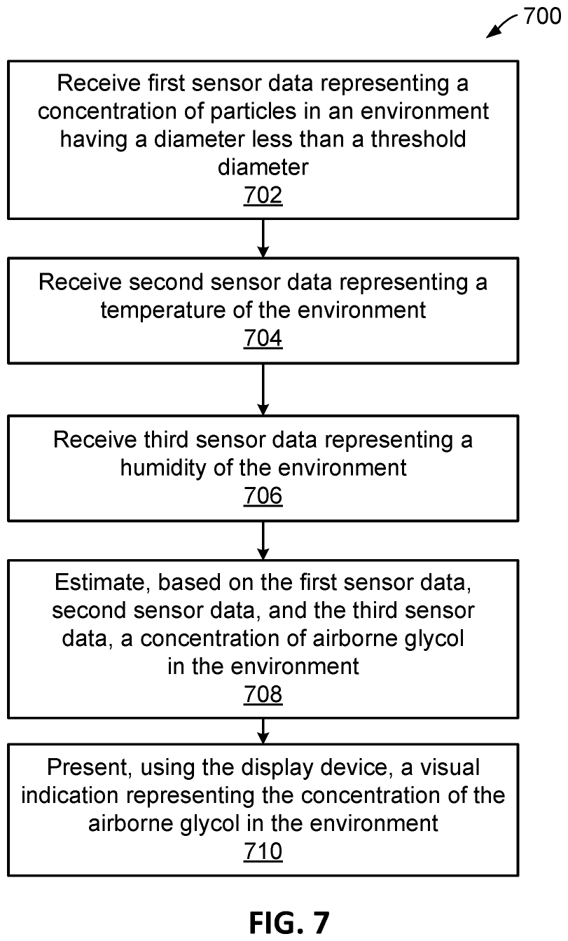

An example process 700 for detecting a presence of and/or a concentration of airborne glycol in an environment is shown in FIG. 7. Some or all of the process 700 can be performed, for example, by the glycol detection system 120 shown and described herein.

In the process 700, a system receives first sensor data representing a concentration of particles in an environment having a diameter less than a threshold diameter (block 702). In some implementations, the threshold diameter can be 10 μm. In some implementations, the threshold diameter can be 2.5 μm.

Further, the system receives second sensor data representing a temperature of the environment (block 704).

Further, the system receives third sensor data representing a humidity of the environment (block 706).

Further, the system estimates, based on the first sensor data, second sensor data, and the third sensor data, a concentration of airborne glycol in the environment (block 708). In some implementations, the airborne glycol can include triethylene glycol (TEG) and/or propylene glycol (PG). For example, the airborne glycol can include vaporized TEG, aerosolized TEG, vaporized PG, and and/or aerosolized PG.

In some implementations, the concentration of the airborne glycol in the environment can be estimated, at least in part, by multiplying the first sensor data by one or more calibration factors to obtain first calibrated sensor data, and estimating the concentration of the airborne glycol in the environment based on the first calibrated sensor data.

The one or more calibration factors can represent a difference between an output of the first sensor and an output of a reference sensor configured to detect a concentration of particles in a reference environment having a diameter less than the threshold diameter. In some implementations, the one or more calibration factors can include a first calibration factor having a value of 0.23 or 0.4, and a second calibration factor having a value of 0.00075.

Further, the concentration of the airborne glycol in the environment can be estimated, at least in part, by (i) determining an adjustment value based on the second sensor data and the third sensor data, (ii) multiplying the adjustment value by an additional calibration factor to obtain a calibrated adjustment value, (iii) determining a sum of the first calibrated sensor data and the calibrated adjustment value, and (iv) estimating the concentration of the airborne glycol in the environment based on the sum of the first calibrated sensor data and the calibrated adjustment value.

Further, the concentration of the airborne glycol in the environment can be estimated, at least in part, by determining that the sum of the first calibrated sensor data and the calibrated adjustment value is the concentration of the airborne glycol in the environment.

In some implementations, the airborne glycol can include an aerosolized portion of the glycol, and a vaporized portion of the glycol. The first calibrated sensor data can represent a concentration of the aerosolized portion of the glycol in the environment, and the calibrated adjustment value can represent a concentration of the vaporized portion of the glycol in the environment.

In some implementations, the additional calibration factor can be 0.50 mg/m³.

In some implementations, the additional calibration factor can be in a range from 0.4 mg/m³ to 1.0 mg/m³.

In some implementations, the additional calibration factor can be in a range from 0.5 mg/m³ to 0.7 mg/m³.

In some implementations, the adjustment value can increase with an increase in the temperature of the environment. Further, the adjustment value can decrease with an increase in the humidity of the environment.

Further, the system presents, using the display device, a visual indication representing the concentration of the airborne glycol in the environment (block 710).

In some implementations, presenting the visual indication representing the concentration of the airborne glycol in the environment can include (i) presenting a first visual indication responsive to determining that the concentration of the airborne glycol in the environment is less than a first threshold concentration, (ii) presenting a second visual indication responsive to determining that the concentration of the airborne glycol in the environment is greater than or equal to the first threshold concentration and less than a second threshold concentration, and/or (iii) presenting a third visual indication responsive to determining that the concentration of the airborne glycol in the environment is greater than or equal to a third threshold concentration.

In some implementations, the first threshold concentration can be 0.4 mg/m³ and the second threshold concentration can be 9.0 mg/m³.

In some implementations, the system can additionally receive a user input comprising instructions to tare at least one of the first sensor data, the second sensor data, or the third sensor data, and in response, tare at least one of the first sensor data, the second sensor data, or the third sensor data.

In some implementations, the system can additionally transmit, to a communications network, at least one of the first sensor data, the second sensor data, the third sensor data, or data representing the concentration of the airborne glycol in the environment (e.g., using a wireless transmitter). As an example, the system can transmit, to a glycol dispersal system, at least one of the first sensor data, the second sensor data, the third sensor data, or data representing the concentration of the airborne glycol. Further, the glycol dispersal system can control a dispersal of glycol into the environment based on the data (e.g., control the frequency at which glycol is dispersed into the environment and/or the amount of glycol that is dispersed into the environment at a time).

In some implementations, the airborne glycol is not visible to a human eye (e.g., an unaided human eye).

### Example Computer System

Some implementations of subject matter and operations described in this specification can be implemented in digital electronic circuitry, or in computer software, firmware, or hardware, including the structures disclosed in this specification and their structural equivalents, or in combinations of one or more of them. For example, in some implementations, the glycol detection system 120 can be implemented at least in part using digital electronic circuitry, or in computer software, firmware, or hardware, or in combinations of one or more of them. In another example, the process 700 can be implemented using digital electronic circuitry, or in computer software, firmware, or hardware, or in combinations of one or more of them.

Some implementations described in this specification can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD-ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

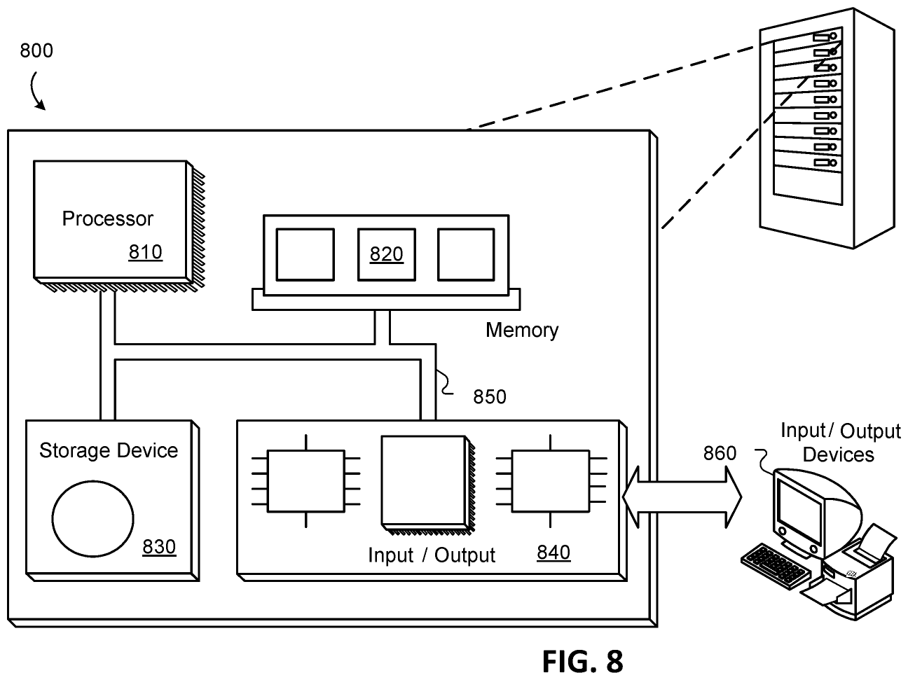

FIG. 8 shows an example computer system 800 that includes a processor 810, a memory 820, a storage device 830 and an input/output device 840. Each of the components 810, 820, 830 and 840 can be interconnected, for example, by a system bus 850. The processor 810 is capable of processing instructions for execution within the system 800. In some implementations, the processor 810 is a single-threaded processor, a multi-threaded processor, or another type of processor. The processor 810 is capable of processing instructions stored in the memory 820 or on the storage device 830. The memory 820 and the storage device 830 can store information within the system 800.

The input/output device 840 provides input/output operations for the system 800. In some implementations, the input/output device 840 can include one or more of a network interface device, e.g., an Ethernet card, a serial communication device, e.g., an RS-232 port, and/or a wireless interface device, e.g., an 802.11 card, a 3G wireless modem, a 4G wireless modem, a 5G wireless modem, etc. In some implementations, the input/output device can include driver devices configured to receive input data and send output data to other input/output devices, e.g., keyboard, printer and display devices 860. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of implementations have been described. Nevertheless, it will be understood that various modifications may be made without departing from the spirit and scope of the invention. Accordingly, other implementations are within the scope of the following claims.

1. A system for estimating a concentration of an airborne glycol in an environment, the system comprising:
a first sensor configured to generate first sensor data representing a concentration of particles in the environment having a diameter less than a threshold diameter;
a second sensor configured to generate second sensor data representing a temperature of the environment;
a third sensor configured to generate third sensor data representing a humidity of the environment;
a display device; and
one or more processors communicatively coupled to the first sensor, the second sensor, the third sensor, and the display device, wherein the one or more processors are configured to:
receive the first sensor data, the second sensor data, and the third sensor data,
estimate, based on the first sensor data, second sensor data, and the third sensor data, the concentration of the airborne glycol in the environment, and
present, using the display device, a visual indication representing the concentration of the airborne glycol in the environment.

2. The system of claim 1, wherein the airborne glycol comprises triethylene glycol (TEG) and propylene glycol (PG).

3. The system of claim 1, wherein the airborne glycol comprises:
vaporized TEG,
aerosolized TEG,
vaporized PG, and
aerosolized PG.

4. The system of claim 1, wherein the threshold diameter is 10 μm.

5. The system of claim 1, wherein the threshold diameter is 2.5 μm.

6. The system of claim 1, wherein presenting the visual indication representing the concentration of the airborne glycol in the environment comprises at least one of:
presenting a first visual indication responsive to determining that the concentration of the airborne glycol in the environment is less than a first threshold concentration,
presenting a second visual indication responsive to determining that the concentration of the airborne glycol in the environment is greater than or equal to the first threshold concentration and less than a second threshold concentration, and
presenting a third visual indication responsive to determining that the concentration of the airborne glycol in the environment is greater than or equal to a third threshold concentration.

7. The system of claim 6, wherein the first threshold concentration is 0.4 mg/m³ and the second threshold concentration is 9.0 mg/m³.

8. The system of claim 1, wherein estimating the concentration of the airborne glycol in the environment comprises:
multiplying the first sensor data by one or more calibration factors to obtain first calibrated sensor data, and
estimating the concentration of the airborne glycol in the environment based on the first calibrated sensor data.

9. The system of claim 8, wherein the one or more calibration factors represent a difference between an output of the first sensor and an output of a reference sensor configured to detect a concentration of particles in a reference environment having a diameter less than the threshold diameter.

10. The system of claim 8, wherein the one or more calibration factors comprises:
a first calibration factor having a value of 0.23 or 0.4, and
a second calibration factor having a value of 0.00075.

11. The system of claim 8, wherein estimating the concentration of the airborne glycol in the environment comprises:
determining an adjustment value based on the second sensor data and the third sensor data,
multiplying the adjustment value by an additional calibration factor to obtain a calibrated adjustment value,
determining a sum of the first calibrated sensor data and the calibrated adjustment value, and
estimating the concentration of the airborne glycol in the environment based on the sum of the first calibrated sensor data and the calibrated adjustment value.

12. The system of claim 11, wherein estimating the concentration of the airborne glycol in the environment comprises:
determining that the sum of the first calibrated sensor data and the calibrated adjustment value is the concentration of the airborne glycol in the environment.

13. The system of claim 11, wherein the airborne glycol comprises an aerosolized portion of the glycol, and a vaporized portion of the glycol and,
wherein the first calibrated sensor data represents a concentration of the aerosolized portion of the glycol in the environment, and
wherein the calibrated adjustment value represents a concentration of the vaporized portion of the glycol in the environment.

14. The system of claim 11, wherein the additional calibration factor is 0.50 mg/m³.

15. The system of claim 11, wherein the additional calibration factor is in a range from 0.4 mg/m³ to 1.0 mg/m³.

16. The system of claim 11, wherein the additional calibration factor is in a range from 0.5 mg/m³ to 0.7 mg/m³.

17. The system of claim 11, wherein the adjustment value increases with an increase in the temperature of the environment.

18. The system of claim 11, wherein the adjustment value decreases with an increase in the humidity of the environment.

19. The system of claim 1, wherein the one or more processors are configured to:
receive a user input comprising instructions to tare at least one of the first sensor data, the second sensor data, or the third sensor data, and
responsive too receiving the user input, tare at least one of the first sensor data, the second sensor data, or the third sensor data.

20. The system of claim 1, wherein the system further comprises a transmitter configured to transmit, to a communications network, at least one of:
the first sensor data,
the second sensor data,
the third sensor data, or
data representing the concentration of the airborne glycol in the environment.

21. The system of claim 18, wherein the transmitter comprises a wireless transmitter.

22. The system of claim 18, wherein the transmitter is configured to transmit, to a glycol dispersal system via the communications network, at least one of:
the first sensor data,
the second sensor data,
the third sensor data, or
the data representing the concentration of the airborne glycol in the environment, and
wherein the glycol dispersal system is configured to regulate the concentration of the airborne glycol in the environment based on at least one of the first sensor data, the second sensor data, the third sensor data, or the data representing the concentration of the airborne glycol in the environment.

23. The system of claim 1, further comprising a housing, wherein the housing at least partially encloses the first sensor, the second sensor, and third sensor, and the one or more processors.

24. The system of claim 23, further comprising a power supply at least partially enclosed by the housing.

25. The system of claim 23, further comprising a connector configured to electrically couple the system to an external power supply.

26. The systems of claim 1, wherein the display device comprises at least one of:
one or more indicator lights, or
a display panel.

27. A method comprising:
receiving first sensor data representing a concentration of particles in an environment having a diameter less than a threshold diameter;
receiving second sensor data representing a temperature of the environment;
receiving third sensor data representing a humidity of the environment;
estimating, based on the first sensor data, second sensor data, and the third sensor data, a concentration of airborne glycol in the environment; and
presenting, using the display device, a visual indication representing the concentration of the airborne glycol in the environment.

28.-49. (canceled)
